(19) United States
(12) Patent Application Publication
(10) Pub. No.: US 2020/0384226 A1
(43) Pub. Date: Dec. 10, 2020

(54) NASOPHARYNGEAL CATHETERS AND APPLICATIONS OF SAME

(71) Applicant: Vanderbilt University, Nashville, TN (US)

(72) Inventors: Michael HIGGINS, Nashville, TN (US); Bret ALVIS, Nashville, TN (US); Doug HESTER, Nashville, TN (US); Adam KING, Nashville, TN (US)

(21) Appl. No.: 16/492,337

(22) PCT Filed: Mar. 9, 2018
(86) PCT No.: PCT/US2018/021661

§ 371 Date: Sep. 9, 2019

Related U.S. Application Data
(60) Provisional application No. 62/469,750, filed on Mar. 10, 2017.
(60) Provisional application No. 62/561,727, filed on Sep. 22, 2017.

(51) Int. Cl. A61M 16/04; A61M 16/00; A61M 16/06; A61M 16/20; A61M 16/08

(52) U.S. Cl. CPC A61M 16/0461; A61M 16/0003; A61M 16/0672; A61M 16/201; A61M 2202/0208; A61M 16/085; A61M 2205/02; A61M 2205/3324; A61M 2230/432; A61M 16/0463

(57) ABSTRACT

A nasopharyngeal catheter for administration of oxygen and monitoring for a patient includes a first catheter having a first lumen, and a second catheter having a second lumen. The first end of the first catheter is operably connected to an oxygen source, the second end of the first catheter operably runs to the posterior oropharynx of the patient, and the first lumen is adapted for oxygen delivery. The second catheter is attached to the first catheter, and the second lumen is adapted for capnography monitoring. The nasopharyngeal catheter further includes an in-line pressure release valve placed proximate to the first end of the first catheter for monitoring pressure of a patient. The nasopharyngeal catheter may also include a pH monitoring sensor for monitoring breath and risk of gastric content translocation of a patient.

### CROSS-REFERENCE TO RELATED PATENT APPLICATIONS

This application claims priority to and the benefit of, pursuant to 35 U.S.C. § 119(e), U.S. provisional patent application Ser. Nos. 62/469,750 and 62/561,727, filed Mar. 10, 2017 and Sep. 22, 2017, respectively, which are incorporated herein by reference in their entireties.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference is individually incorporated by reference. In terms of notation, hereinafter, [n] represents the nth reference cited in the reference list. For example, [1] represents the first reference cited in the reference list, namely, D. S. Dark, D. R. Campbell, and L. J. Wesselius, “Arterial oxygen desaturation during gastrointestinal endoscopy,” Am J Gastroenterol, vol. 85, pp. 1317-21, October 1990.

### FIELD

The present disclosure relates generally to administration of oxygen to a patient, and more particularly to nasopharyngeal catheters and applications of the same.

### BACKGROUND

The background description provided herein is for the purpose of generally presenting the context of the present disclosure. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the present disclosure.

The administration of oxygen is standard of care during and after general anesthetics. There are many methods that this can be accomplished, some better than others. Recent evidence has shown that the administration of oxygen through a nasopharyngeal catheter is superior to the current practice of nasal cannula, oxygen face tent, oxygen face mask, etc. For example, the administration of oxygen with these nasopharyngeal catheters was evaluated in morbidly obese patients to prevent desaturations prior to direct laryngoscopy. It is found that this technique helps prevent oxygen desaturation for four minutes compared to 145 seconds in the control group. This prolonged time provided when there was insufflation of oxygen with a nasopharyngeal catheter was confirmed. These catheters are found to improve obstruction, improve hemoglobin saturation, and prolong the period to desaturation during apnea. However, to date, these catheters have not been adapted with capnography monitoring. Also, there are case-reports of insufflation injury from pressure build up within the esophagus of a patient. However, to date, these catheters have not been adapted with pressure relief technology. During sedation translocation of gastric content from stomach to lungs is a major morbidity that has been reported and studied. However, to date, these catheters nor any supraglottic airway device has been equipped with pH or impedence monitoring.

Therefore, a heretofore unaddressed need exists in the art to address the aforementioned deficiencies and inadequacies.

### SUMMARY

In one aspect, the invention relates to a nasopharyngeal catheter for administration of oxygen and monitoring for a patient. In one embodiment, the nasopharyngeal catheter includes a first catheter having a first end, a second end and a first lumen defined therebetween, wherein the first lumen is adapted for oxygen delivery; and a second catheter having a first end, a second end and a second lumen defined therebetween, wherein the second catheter is attached to the first catheter, and the second lumen is adapted for capnography monitoring.

In one embodiment, the first catheter is formed of a flexible material including silicon or a siliconized material.

In one embodiment, the first lumen has a size of about 10 Fr.

In one embodiment, the first end of the first catheter is operably connected to an oxygen source, and the second end of the first catheter operably runs to the posterior oropharynx of the patient.

In one embodiment, the first catheter has a plurality of holes defined proximal to the second end of the first catheter and being in fluidic communication with the first lumen.

In one embodiment, the first end of the second catheter is operably connected to a detector, and the second end of the second catheter is positioned in a desired distance from the second end of the first catheter. In one embodiment, the desired distance is about 1 inch. In one embodiment, the detector is an end-tidal capnography.

In one embodiment, the second catheter comprises one or more sensors placed inside the second lumen for monitoring end-tidal CO₂ (ETCO2).

In one embodiment, the nasopharyngeal catheter further includes an in-line pressure release valve placed proximate to the first end of the first catheter and being in fluidic communication with the first lumen.

In one embodiment, the in-line pressure release valve is a t-piece pressure release valve that operably opens if there is a buildup of a predetermined pressure in the first lumen.

In one embodiment, the predetermined pressure about 30 cm H₂O.

In one embodiment, the pressure that the valve will operably open can be set by clinician.

In one embodiment, the nasopharyngeal catheter further includes a pH monitoring sensor for monitoring breath and risk of gastric content translocation of the patient. In one embodiment, the pH monitoring sensor is placed inside the first lumen at a distance from the second end of the first catheter. In another embodiment, the pH monitoring sensor is placed the second end of the first catheter.

In another aspect, the invention relates to a nasopharyngeal catheter for administration of oxygen and monitoring for a patient. In one embodiment, the nasopharyngeal catheter includes a first catheter having a first end, a second end and a first lumen defined therebetween; and an in-line pressure release valve placed proximate to the first end of the first catheter and being in fluidic communication with the first lumen. The first end of the first catheter is operably connected to an oxygen source; the second end of the first catheter operably runs to the posterior oropharynx of the patient; and the first lumen is adapted for oxygen delivery.

In one embodiment, the first catheter has a plurality of holes defined proximal to the second end of the first catheter and being in fluidic communication with the first lumen.

In one embodiment, the in-line pressure release valve is a t-piece pressure release valve that operably opens if there is a buildup of a predetermined pressure in the first lumen. In one embodiment, the predetermined pressure about 30 cm H₂O.

In one embodiment, the nasopharyngeal catheter further includes a pH monitoring sensor for monitoring breath of the patient. In one embodiment, the pH monitoring sensor is placed inside the first lumen at a distance from the second end of the first catheter. In another embodiment, the pH monitoring sensor is placed the second end of the first catheter.

In certain aspects, the invention also relates to a system for administration of oxygen and monitoring for a patient, comprising the nasopharyngeal catheter as disclosed above.

In one embodiment, the system is connectable to a nasal cannula that operably allows a turn-and-lock approach to connecting the nasopharyngeal catheter.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows schematically a nasopharyngeal catheter according to one embodiment of the present invention.

FIG. 2 shows schematically a nasopharyngeal catheter according to another embodiment of the present invention.

FIG. 3 shows schematically a nasopharyngeal catheter according to yet another embodiment of the present invention.

FIG. 4 shows schematically a nasopharyngeal catheter according to further embodiment of the present invention.

### DETAILED DESCRIPTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art Like reference numerals refer to like elements throughout. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more”. The word “exemplary” is used herein to mean “serving as an example, instance, or illustration”. Any aspect described herein as “exemplary” is not necessarily to be construed as preferred or advantageous over other aspects. Unless specifically stated otherwise, the term “some” refers to one or more. Combinations such as “at least one of A, B, or C”, “one or more of A, B, or C”, “at least one of A, B, and C”, “one or more of A, B, and C”, and “A, B, C, or any combination thereof” include any combination of A, B, and/or C, and may include multiples of A, multiples of B, or multiples of C. Specifically, combinations such as “at least one of A, B, or C”, “one or more of A, B, or C”, “at least one of A, B, and C”, “one or more of A, B, and C”, and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module”, “mechanism”, “element”, “device” and the like may not be a substitute for the word “means”. As such, no claim element is to be construed as a means plus function unless the element is expressly recited using the phrase “means for”. It should also be understood that one or more steps within a method may be executed in different order (or concurrently) without altering the principles of the invention.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term are the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the disclosure.

Furthermore, relative terms, such as “lower” or “bottom” and “upper” or “top”, may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, the terms “comprise” or “comprising”, “include” or “including”, “carry” or “carrying”, “has/have” or “having”, “contain” or “containing”, “involve” or “involving” and the like are to be understood to be open-ended, i.e., to mean including but not limited to.

Typically, terms such as “about,” “approximately,” “generally,” “substantially,” and the like unless otherwise indicated mean within 20 percent, preferably within 10 percent, preferably within 5 percent, and even more preferably within 3 percent of a given value or range. Numerical quantities given herein are approximate, meaning that the term “about,” “approximately,” “generally,” or “substantially” can be inferred if not expressly stated.

The description is now made as to the embodiments of the invention in conjunction with the accompanying drawings. It should be understood that specific embodiments described herein are merely intended to explain the invention, but not intended to limit the invention. Various modifications to these aspects will be readily apparent to those skilled in the art, and the generic principles defined herein may be applied to other aspects.

In accordance with the purposes of this invention, as embodied and broadly described herein, this invention relates to a nasopharyngeal catheter for oxygen administration and capnography monitoring for a patient.

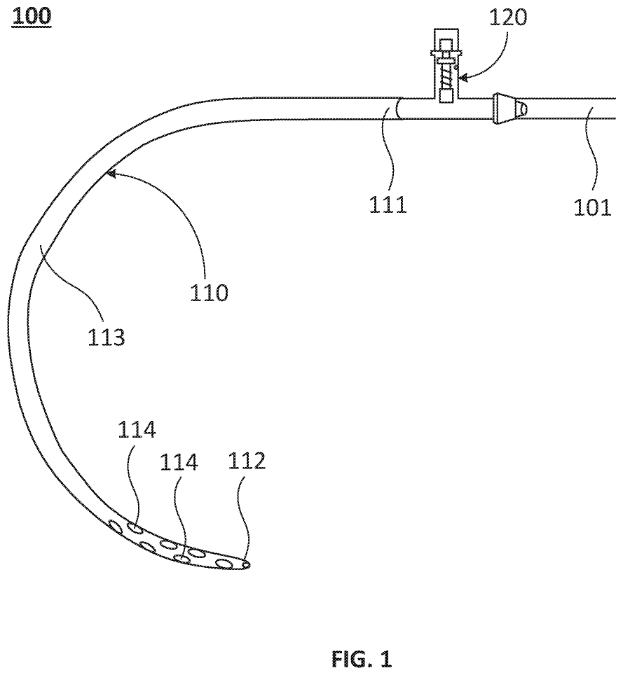

Referring to FIG. 1, a nasopharyngeal catheter for administration of oxygen and monitoring of pressure for a patient is schematically shown according to one embodiment of the invention. In this exemplary embodiment, the nasopharyngeal catheter 100 includes a first catheter 100 having a first end 111, a second end 112 and a first lumen 113 defined therebetween. The first end 111 of the first catheter 110 is operably connected to an oxygen source 101; the second end 112 of the first catheter 110 operably runs to the posterior oropharynx of the patient (not shown). The first lumen 113 is an oxygen lumen adapted for oxygen delivery. The first catheter 110 has a plurality of holes 114 defined proximal to the second end 112 of the first catheter 110 and being in fluidic communication with the first lumen 113.

In some embodiments, the first lumen 113 has a size of about 10 Fr.

In some embodiments, the first catheter 110 is formed of a flexible material including silicon or a siliconized material.

The nasopharyngeal catheter 100 also includes an in-line pressure release valve 120 that is placed proximate to the first end 111 of the first catheter 110 and in fluidic communication with the first lumen 113.

In one embodiment, the in-line pressure release valve 120 is a t-piece pressure release valve that operably opens if there is a buildup of a predetermined pressure in the first lumen 113. The predetermined pressure is adjustable. In one embodiment, the predetermined pressure about 30 cm H₂O. The predetermined pressure can also be other values depending upon its applications.

In some embodiments, the nasopharyngeal catheter further includes a pH monitoring sensor for monitoring breath of the patient. In one embodiment, the pH monitoring sensor is placed inside the first lumen at a distance from the second end of the first catheter. In another embodiment, the pH monitoring sensor is placed the second end of the first catheter.

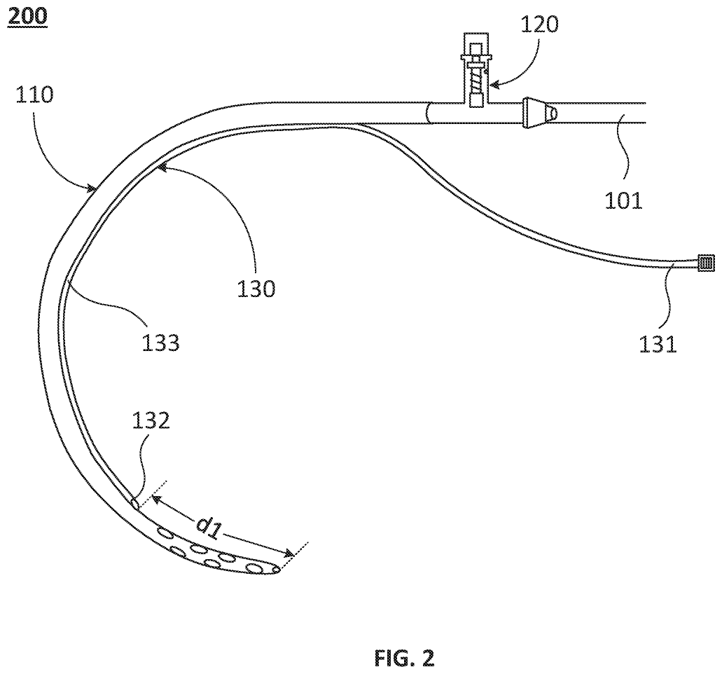

FIGS. 2-4 respectively show a schematic nasopharyngeal catheter for administration of oxygen and monitoring of capnography, pressure and/or pH for a patient is shown according to various embodiments of the invention

As shown in FIG. 2, the nasopharyngeal catheter 200 includes a first catheter 110 and a second catheter 130 attached to the first catheter 110. The first catheter 110 is the same as that shown in FIG. 1 and disclosed above.

The second catheter 130 has a first end 131, a second end 132 and a second lumen 122 defined therebetween. The second lumen 133 is a dedicated lumen for capnography monitoring which runs along the first lumen (oxygen lumen) to help a clinician operably monitor ventilation of a patient.

Operably, the first end 131 of the second catheter 130 is connected to a detector, while the second end 132 of the second catheter 130 is positioned in a desired distance, d1, from the second end 112 of the first catheter 110. In certain embodiments, the detector is an end-tidal capnography for monitoring end-tidal CO₂ (ETCO2). In certain embodiments, the desired distance d1 is about 1 inch.

In certain embodiments, the second catheter 130 may have one or more sensors (not shown) placed inside the second lumen 133 for monitoring the ETCO2.

Furthermore, the nasopharyngeal catheter 200 may also include an in-line pressure release valve 120 that is placed proximate to the first end 111 of the first catheter 110 and in fluidic communication with the first lumen 113. In some embodiments, the in-line pressure release valve 120 is a t-piece pressure release valve (a pop-off valve) that operably opens if there is a buildup of a predetermined pressure in the first lumen 113. In some embodiments, the predetermined pressure about 30 cm H₂O. The pop-off valve is adapted to manage the previous concerns for esophagus perforation due to increased pressure.

Accordingly, the double lumen nasopharyngeal oxygen catheter with a pop-off valve capability allows the administration of oxygen to a patient, monitoring of end-tidal CO₂, and has the ability of pressure release if there is a buildup of pressure within the patient.

There is also a subset of patients that these catheters' pH monitoring capabilities would allow for clinicians to identify regurgitation and intervene with suction or incubation. A pH monitor sensor can be adapted if the clinician feels it necessary to monitor for possible gastric regurgitation.

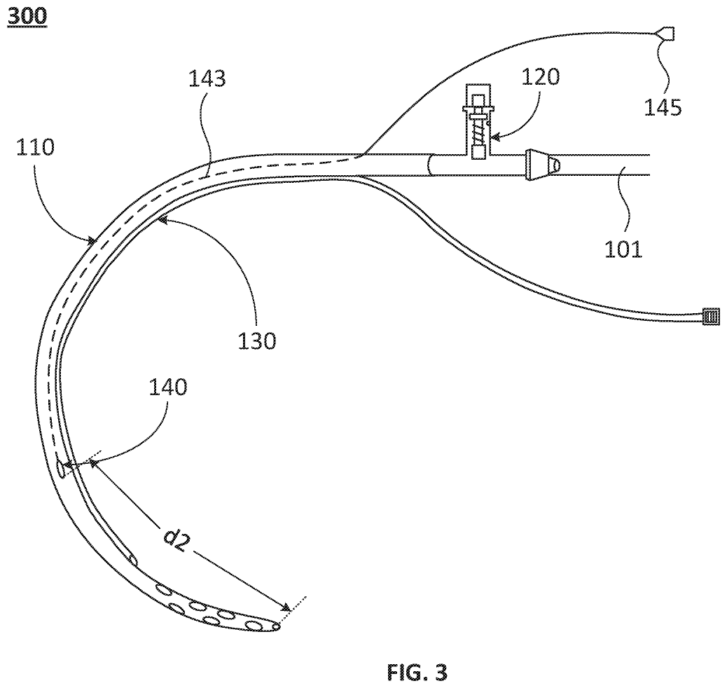

As shown in FIG. 3, in addition to a first catheter 110, a second catheter 130 and a in-line pressure release valve 120 shown in FIG. 2, the nasopharyngeal catheter 300 further includes a pH monitoring sensor 140 for monitoring continuous monitoring for aspiration of a patient. In this exemplary embodiment shown in FIG. 3, the pH monitoring sensor 140 is placed inside the first lumen 113 at a distance, d2, from the second end 112 (which is operably placed in the oropharynx of a patient) of the first catheter 110. The pH monitoring sensor 140 is operably connected to a pH monitor 145 through a pH monitoring line 143. The pH monitoring line 143 may be adapted along with the first catheter 110.

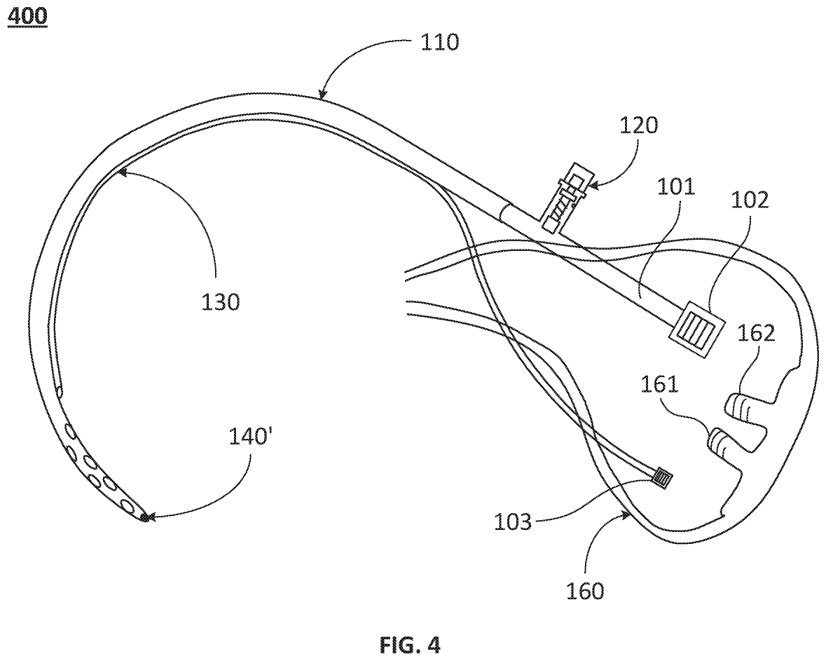

In another embodiment as shown in FIG. 4, the pH monitoring sensor 140′ is placed the second end 112 of the first catheter 110.

Altogether, the nasopharyngeal catheter according embodiments of the invention provides a clinician a safer and more effective way to administer oxygen and monitor their patients. This safe, well tolerated and more effective device allows for supraglottic oxygen administration, and capnography, pressure and/or pH monitoring.

In another aspect, the invention relates to a system for oxygen administration and capnography monitoring, and pH monitoring for a patient, comprising the nasopharyngeal catheter as disclosed above.

In one embodiment, as shown in FIG. 4, the system 400 is connectable to a special designed nasal cannula 160 that operably allows a “turn and lock” approach to connecting the nasopharyngeal catheter. The special designed nasal cannula 160 has a capnography nasal cannula hub 161 operably connectable to the capnography attachment hub 103, which is connected to the first end 131 of the capnography monitoring lumen 130, and an oxygen nasal cannula hub 162 operably connectable to the oxygen attachment hub 102, which is connected to the oxygen source 101.

Among other things, the invented nasopharyngeal catheter has the following advantages: more affective oxygen administration compared with the nasal cannula or face mask, concomitant monitoring of capnography to confirm ventilation with combined oxygen administration to a sedated patient, pH monitoring to warn clinicians of the presence of gastric content reaching the posterior oropharynx, and pressure release in the event of unsafe esophageal pressure build up.

Accordingly to the invention, oxygen supplementation via the invented nasopharyngeal catheter decrease the incidence of oxygen desaturation to less than 92% in patients undergoing intravenous sedation for orthopedic and oral surgical procedures orthopedic procedures as compared to traditional oxygen supplementation via nasal cannula.

Among other things, applications of the invented nasopharyngeal catheter include, but not limited to, oxygen administration during general anesthetics with the patient spontaneous breathing, oxygen administration post general anesthesia, and oxygen administration during apnea prior to direct laryngoscopy.

Without intent to limit the scope of the invention, examples and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Randomized, Blinded, Proof of Concept Comparison Study of a Novel Double Lumen Nasopharyngeal Catheter Versus Traditional Nasal Cannula During Deep Sedation Gastrointestinal Procedures

### Introduction

Patients undergoing intravenous sedation routinely experience episodes of hypoxemia. In patients undergoing endoscopic procedures with conscious sedation, rates of hypoxemia are been reported to range from about 10 to 57% [1]. These episodes of hypoxemia occur in part because anesthesia induces changes in the muscle tone of the upper airway and may commonly result in airway obstruction [2]. Because oxygen is typically administered by nasal cannula (NC) during such procedures, an episode of hypoxemia may require a physical intervention such as a jaw lift to relieve the airway obstruction by the personnel providing sedation which can interrupt the procedure. As an alternative method for giving supplemental oxygen, nasopharyngeal oxygen has been shown to be a comfortable substitute for face mask for providing oxygen supplementation [3, 4]. Because the nasopharyngeal catheter provides oxygen supplementation immediately supraglottic, it delivers oxygen past the point of airway obstruction that is induced by general anesthesia [5]. It has been typically used in the pediatric patients, intensive care unit or the postoperative period; and it has been shown to require lower oxygen flow rates to achieve the same oxygenation as facemask or nasal cannula [3, 4, 6-8]. However, its use for patients undergoing intravenous sedation for endoscopic procedures has not been reported.

This exemplary study shows that oxygen supplementation via a nasopharyngeal catheter (NPC) decreases the number of episodes of hypoxemia, defined by an oxygen saturation less than 92%, as compared with traditional NC oxygen supplementation in patients undergoing intravenous sedation for endoscopic gastrointestinal procedures.

### Materials and Methods

Study Design and population: a blinded randomized control trial was performed, where patients (participants) were assigned randomly to receive supplemental oxygen by either a standard nasal cannula or a nasal pharyngeal catheter. Patients were enrolled if they were receiving a colonoscopy or endoscopy procedure within the gastrointestinal operative suite. Sixty (60) patients were enrolled in this study.

This study was approved by the institutional review board of Vanderbilt University (Nashville, Tenn.) and written informed consent was obtained from all study participants. This study was registered with clinicaltrials.gov (NCT02219464). The study enrolled adult patients undergoing intravenous general anesthesia with propofol for endoscopic gastrointestinal procedures from July 2014 to November 2015. Patients were excluded if they required endotracheal intubation for their procedure, had existing esophageal varices or perforation or were American Society of Anesthesiologists Physical Classification Class 4 or higher. Patients were randomized to oxygen supplementation with nasal cannula (Salter Labs, Arvin, Calif.) or a nasopharyngeal catheter via computer generated allocation in blocks of 10. Randomizations were placed in a sealed envelope by an author (M. S. H.) who was blinded to patient recruitment and data collection.

Airway Management: Either a standard nasal cannula or a nasopharyngeal catheter of the invention was placed in the patient after induction of anesthesia. Sedation consisted of propofol infusion.

Selection and dosing of sedation medications were at the discretion of the attending anesthesiologists. After induction, if the patient was randomized to the nasopharyngeal catheter one of the study authors placed the device keeping the CRNA (Certified Registered Nurse Anesthetist) blinded to the device. Following placement of the airway device, an observer unaware of the patient group assignment came into the operating room to record study data. The device was removed at the end of the procedure. The patient was evaluated immediately postoperatively for any complications.

Statistical Analysis: The primary outcome was in incidence of hypoxemia as defined by oxygen saturation less than about 92%. Based upon clinical observation, it was estimated that oxygen desaturation to less than about 92% occurs in approximately about 80% of patients undergoing intravenous sedation for endoscopic procedures. The sample size of 60 subjects, 30 in each group, was calculated assuming a power of about 0.80 and an alpha of about 0.05 in order to detect an about 30% reduction in the incidence of clinically significant desaturation. Secondary outcomes included the number of airway assist maneuvers such as jaw lift, number of desaturations defined as oxygen saturations less than about 92%, and need for more advanced airway interventions such as oral or nasal airway use, laryngeal mask airway placement or endotracheal intubation. For the purpose of this study, an airway assist maneuver was defined as lifting of the jaw at the mandibular angle.

Study data including outcomes and patient demographics were collected and entered into the REDCap (Research Electronic Data Capture) database hosted at Vanderbilt University [9]. Continuous data were reported as mean+/−standard deviation and were analyzed using a two-tailed Student's t-test with a p<0.05 considered statistically significant. Categorical data were reported as numbers and percentages and analyzed using a two-tailed Fisher's Exact with p<0.05 considered clinically significant. All analyses were performed using SOFA (Statistics Open ForAll) Statistics Version 1.3.0, which is an open-source statistical package.

### Results

Of the 60 enrolled patients; three subjects in the NPC group were excluded from further analysis. There was no difference between group in age, ASA classification, Body Mass Index, oropharyngeal classification or sedation. Patients who received nasopharyngeal oxygen supplementation were less likely to experience a clinically significant oxygen desaturation event 3 of 27 (about 7.5%) versus 12 of 30 subjects (about 32.4%), p=0.013. Interventions to assists with airway management were required for fewer patients in the NPC group 4 (about 10.0%) versus the NC group, 17 (about 45.9%), p=0.001.

Sixty patients were enrolled in the study. Three patients from the NPC group were excluded from the analysis. One subject withdrew consent prior to the procedure, one endoscopy was cancelled due to inadequate bowel preparation, and the third patient was excluded due to NPC catheter malfunction, which was noted prior to insertion into the patient. There were no significant differences between groups in age, gender, American Society of Anesthesiologists Physical Classification score, baseline room air oxygen saturation, body mass index or total propofol dose (Table 1).

TABLE 1
Demographic Information
DEMOGRAPHIC INFORMATION

| | NC | | NPC | | |
|---|---|---|---|---|---|
| | (n = 30) | | (n = 27) | | p-value |
| Age | 54.5 ± 16.2 | | 55.2 ± 16.4 | | 0.88 |
| Gender | | | | | 0.65 |
| Male (%) | 16 | (53.3) | 16 | (59.3) | |
| Female (%) | 14 | (46.7) | 11 | (40.7) | |
| ASA | | | | | 0.12 |
| Classification | | | | | |
| 2(%) | 15 | (50.0) | 8 | (29.6) | |
| 3 (%) | 15 | (50.0) | 19 | (70.3) | |
| BMI | 29.7 ± 6.0 | | 31.8 ± 9.7 | | 0.32 |
| Mallampati | | | | | 0.46 |
| Classification | | | | | |
| 1 (%) | 4 | (13.3) | 3 | (11.1) | |
| 2 (%) | 15 | (50.0) | 11 | (40.7) | |
| 3 (%) | 11 | (36.7) | 11 | (40.7) | |
| 4 (%) | 0 | (0.0) | 2 | (7.5) | |
| Room Air Oxygen | 98.5 ± 1.7 | | 97.7 ± 2.7 | | 0.19 |
| Saturation | | | | | |
| Procedure Length | 20.0 ± 15.6 | | 25.6 ± 25.7 | | 0.32 |
| (min) | | | | | |
| Sedation | | | | | |
| Fentanyl (mcg) | 65.9 ± 28.0 | | 67.8 ± 33.9 | | 0.83 |
| Maximum | 212.0 ± 49.4 | | 200.7 ± 27.1 | | 0.30 |
| Propofol | | | | | |
| Infusion rate | | | | | |
| (mcg/kg/min) | | | | | |
| Total Propofol | 432.6 ± 250.8 | | 549.9 ± 412.5 | | 0.20 |
| dose (mg) | | | | | |
| Procedure Type | | | | | 0.46 |

| | | |
|---|---|---|
| Upper | 20 | 14 |
| Endoscopy | | |
| Lower | 9 | 12 |
| Endoscopy | | |
| Combined | 2 | 4 |
| Upper/Lower | | |
| Endoscopy | | |

(NC = nasal cannula, NPC = nasopharyngeal catheter, ASA = American Society of Anesthesiologists)

Patients in the NPC group were less likely to experience an episode of oxygen desaturation less than about 92%, 3 (about 11.1%) versus 12 (about 40%), (p=0.013) in the NC group. Patients in the NPC group were also less likely to require an airway assist maneuver, 4 (about 14.8%) versus 17 (about 56.7%), (p=0.001) (Table 2). The NPC group included about 12 upper endoscopies and 11 lower colonoscopies versus 18 and 8 in the NC group, respectively (p=0.46) (Table 1). No patients in either group required more advanced airway interventions such as an oral or nasal airway, laryngeal mask airway (LMA) placement or endotracheal intubation. One patient in the NPC group experienced a nosebleed that resolved spontaneously. No patients in either group reported discomfort with either device.

TABLE 2
Outcomes
OUTCOMES

| | | |
|---|---|---|
| NC | NPC | |
| (n = 30) | (n = 27) | p-value |

| | | | | | |
|---|---|---|---|---|---|
| Number of patients | 12 | (40.0) | 3 | (11.1) | 0.013 |
| experiencing an | | | | | |
| Oxygen Desaturation < | | | | | |
| 92% (%) | | | | | |

| | | | |
|---|---|---|---|
| Episodes of Oxygen | 0.7 ± 1.2 | 0.1 ± 0.3 | 0.01 |
| Desaturation per | | | |
| patient | | | |

| | | | | | |
|---|---|---|---|---|---|
| Airway Assist | 17 | (56.7) | 4 | (14.8) | 0.001 |
| Maneuver Required | | | | | |
| (%) | | | | | |

| | | | |
|---|---|---|---|
| Max Oxygen Flow rate | 3.9 ± 1.3 | 3.9 ± 1.7 | 0.84 |
| (L/min) | | | |

(NC = nasal cannula, NPC = nasopharyngeal catheter)

### Discussion

The nasal cannula has long been the standard method for oxygen supplementation during anesthesia for endoscopic procedures. Despite routine use of nasal cannula, the incidence of oxygen desaturation is reported to occur in up to about 57% of patients and likely due to upper airway obstruction which is estimated to occur in about 15% of patients [1, 2, 10]. Similarly, in the present study about 40% of patients receiving oxygen by nasal cannula experienced oxygen desaturation less than about 92% and about 17% required physical interventions to relieve airway obstruction. The present study was designed to determine if supplemental oxygen delivered through a novel double lumen catheter below the common location of airway obstruction would reduce the incidence of oxygen desaturation. In the group using a nasopharyngeal catheter we observed an about 29% reduction in episodes of oxygen desaturation and a 42% reduction in the need for a physical maneuver to relieve airway obstruction. NPC use has been reported to be less comfortable than nasal cannula oxygen; however, in this study there were no complaints of postoperative airway pain in either group [4].

While this study was performed with a blinded observer, the in room provider was not blinded to the study device. This may of lead to some bias with management during these cases especially in terms of performing jaw thrusts to relieve airway obstruction. Significant changes in oxygen saturation would of still prompted intervention by the in room provider.

In conclusion, the findings of this study show that NPC oxygen supplementation for adult patients undergoing endoscopic procedures results in less oxygen desaturations and physical interventions for relief of airway obstruction than the nasal cannula. These findings suggest more research should be done to evaluate the use of nasopharyngeal catheters in other arenas of anesthesia or other clinical environments where supplemental oxygen is required in the setting of potential airway obstruction. While a nasal cannula is currently the standard device used for oxygen supplementation during deep sedation, the NPC may be an alternative oxygen delivery device especially in those prone to upper airway obstruction.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments are chosen and described in order to explain the principles of the disclosure and their practical application so as to activate others skilled in the art to utilize the disclosure and various embodiments and with various modifications as are suited to the particular use contemplated. Alternative embodiments will become apparent to those skilled in the art to which the present disclosure pertains without departing from its spirit and scope. Accordingly, the scope of the present disclosure is defined by the appended claims rather than the foregoing description and the exemplary embodiments described therein.

### REFERENCE LIST

[1] D. S. Dark, D. R. Campbell, and L. J. Wesselius, “Arterial oxygen desaturation during gastrointestinal endoscopy,” Am J Gastroenterol, vol. 85, pp. 1317-21, October 1990.
[2] D. R. Hillman, P. R. Platt, and P. R. Eastwood, “The upper airway during anaesthesia,” Br J Anaesth, vol. 91, pp. 31-9, July 2003.
[3] G. M. Eastwood and M. J. Dennis, “Nasopharyngeal oxygen (NPO) as a safe and comfortable alternative to face mask oxygen therapy,” AustCrit Care, vol. 19, pp. 22-4, February 2006.
[4] G. M. Eastwood, B. O'Connell, A. Gardner, and J. Considine, “Evaluation of nasopharyngeal oxygen, nasal prongs and facemask oxygen therapy devices in adult patients: a randomised crossover trial,” Anaesth Intensive Care, vol. 36, pp. 691-4, September 2008.
[5] A. B. King A B, Hester D L, Higgins M S, “Treating Acute Oxygen Desaturation during deep sedation with a novel double lumen nasopharyngeal catheter,” 2015.
[6] G. M. Eastwood, J. H. Reeves, and B. S. Cowie, “Nasopharyngeal oxygen in adult intensive care-lower flows and increased comfort,” Anaesth Intensive Care, vol. 32, pp. 670-1, October 2004.
[7] M. W. Weber, A. Palmer, A. Oparaugo, and E. K. Mulholland, “Comparison of nasal prongs and nasopharyngeal catheter for the delivery of oxygen in children with hypoxemia because of a lower respiratory tract infection,” J Pediatr, vol. 127, pp. 378-83, September 1995.
[8] K. Stausholm, S. Rosenberg-Adamsen, M. Shiver, H. Kehlet, and J. Rosenberg, “Comparison of three devices for oxygen administration in the late postoperative period,” Br J Anaesth, vol. 74, pp. 607-9, May 1995.
[9] P. A. Harris, R. Taylor, R. Thielke, J. Payne, N. Gonzalez, and J. G. Conde, “Research electronic data capture (REDCap)—A metadata-driven methodology and workflow process for providing translational research informatics support,” Journal of Biomedical Informatics, vol. 42, pp. 377-381, 4//2009.
[10] J. R. Miner, W. Heegaard, and D. Plummer, “End-tidal Carbon Dioxide Monitoring during Procedural Sedation,” Academic Emergency Medicine, vol. 9, pp. 275-280, 2002.

1. A nasopharyngeal catheter for administration of oxygen and monitoring for a patient, comprising:
a first catheter having a first end, a second end and a first lumen defined therebetween, wherein the first lumen is adapted for oxygen delivery; and
a second catheter having a first end, a second end and a second lumen defined therebetween, wherein the second catheter is attached to the first catheter, and the second lumen is adapted for capnography monitoring.

2. The nasopharyngeal catheter of claim 1, wherein the first catheter is formed of a flexible material including silicon or a siliconized material.

3. The nasopharyngeal catheter of claim 1, wherein the first lumen has a size of about 10 Fr.

4. The nasopharyngeal catheter of claim 1, wherein the first end of the first catheter is operably connected to an oxygen source, and the second end of the first catheter operably runs to the posterior oropharynx of the patient.

5. The nasopharyngeal catheter of claim 4, wherein the first end of the second catheter is operably connected to a detector, and the second end of the second catheter is positioned in a desired distance from the second end of the first catheter.

6. The nasopharyngeal catheter of claim 5, wherein the desired distance is about 1 inch.

7. The nasopharyngeal catheter of claim 5, wherein the detector is an end-tidal capnography.

8. The nasopharyngeal catheter of claim 1, wherein the second catheter comprises one or more sensors placed inside the second lumen for monitoring end-tidal CO₂ (ETCO2).

9. The nasopharyngeal catheter of claim 1, further comprising an in-line pressure release valve placed proximate to the first end of the first catheter and being in fluidic communication with the first lumen.

10. A system for administration of oxygen and monitoring for a patient, comprising the nasopharyngeal catheter of claim 1.

11. The system of claim 10, being connectable to a nasal cannula that operably allows a turn-and-lock approach to connecting the nasopharyngeal catheter.

12. A nasopharyngeal catheter for administration of oxygen and monitoring for a patient, comprising:
a first catheter having a first end, a second end and a first lumen defined therebetween, wherein the first end of the first catheter is operably connected to an oxygen source, the second end of the first catheter operably runs to the posterior oropharynx of the patient, and the first lumen is adapted for oxygen delivery; and
an in-line pressure release valve placed proximate to the first end of the first catheter and being in fluidic communication with the first lumen.

13. The nasopharyngeal catheter of claim 12, wherein the first catheter has a plurality of holes defined proximal to the second end of the first catheter and being in fluidic communication with the first lumen.

14. The nasopharyngeal catheter of claim 12, wherein the in-line pressure release valve is a t-piece pressure release valve that operably opens if there is a buildup of a predetermined pressure in the first lumen.

15. The nasopharyngeal catheter of claim 14, wherein the predetermined pressure about 30 cm H₂O.

16. The nasopharyngeal catheter of claim 12, further comprising a pH monitoring sensor for monitoring breath and risk of gastric content translocation of the patient.

17. The nasopharyngeal catheter of claim 16, wherein the pH monitoring sensor is placed inside the first lumen at a distance from the second end of the first catheter.

18. The nasopharyngeal catheter of claim 17, wherein the pH monitoring sensor is placed the second end of the first catheter.

19. A system for administration of oxygen and monitoring for a patient, comprising the nasopharyngeal catheter of claim 12.

20. The nasopharyngeal catheter of claim 1, further comprising a pH monitoring sensor for monitoring breath and risk of gastric content translocation of the patient.

21. The nasopharyngeal catheter of claim 4, wherein the first catheter has a plurality of holes defined proximal to the second end of the first catheter and being in fluidic communication with the first lumen.

22. The nasopharyngeal catheter of claim 9, wherein the in-line pressure release valve is a t-piece pressure release valve that operably opens if there is a buildup of a predetermined pressure in the first lumen.
